(19) United States
(12) Patent Application Publication
(10) Pub. No.: US 2026/0183207 A1
(43) Pub. Date: Jul. 2, 2026

(54) COSMETIC METHODS, KIT-OF-PARTS AND USE OF SKIN PREBIOTIC ACTIVE IN COMBINATION WITH ANTIMICROBIAL METAL SALT FOR PROVIDING SKIN CARE BENEFITS BY MICROBIAL REPROFILING OF SKIN

(71) Applicant: Conopco, inc., d/b/a UNILEVER, Hoboken, NJ (US)

(72) Inventors: Joanne Elizabeth HUNT, Wirral (GB); Alexander Gordon JAMES, Higham Ferrers (GB); Barry Gerard MURPHY, Mancot, Deeside (GB)

(73) Assignee: Conopco, inc., d/b/a UNILEVER, Hoboken, NJ (US)

(21) Appl. No.: 19/131,445

(22) PCT Filed: Nov. 2, 2023
(86) PCT No.: PCT/EP2023/080538

§ 371 Date: May 20, 2025

(30) Foreign Application Priority Data
Nov. 21, 2022 (EP) 22208567.2

(51) Int. Cl. A61K 8/26; A61K 8/362; A61Q 15/00; A61Q 17/00; A61Q 19/00

(52) U.S. Cl. CPC A61K 8/26; A61K 8/362; A61Q 15/00; A61Q 17/005; A61Q 19/00; A61K 2800/884

(57) ABSTRACT

The invention provides a cosmetic method for providing skin care benefits by microbial reprofiling of skin; the method comprising the steps of: (i) treating the skin by topical application of an antimicrobial metal salt, followed by (ii) washing the treated skin and treating the washed skin by topical application of a skin prebiotic active. The invention also provides the cosmetic use of a skin prebiotic active, in combination with an antimicrobial metal salt, for providing skin care benefits by microbial reprofiling of skin.

### FIELD OF THE INVENTION

The present invention relates to a cosmetic method for providing skin care benefits by microbial reprofiling of skin.

### BACKGROUND OF THE INVENTION

Human skin harbours many diverse microorganisms, which colonize the stratum corneum of the epidermis and skin appendages such as sweat glands and hair follicles. The total of microorganisms in and on our skin is termed the cutaneous microbiota, and the skin microbiome is their collective genome.

Sequencing studies of diverse skin sites in healthy adults have shown that the composition of the skin microbiome is determined primarily by body site. Microbial colonization is differentially shaped by the physiological and topological variation of the skin, and varies systematically among different skin habitats, such as between dry, moist, and sebaceous skin.

The human axillae (underarms) are covered by squames, hair shafts and follicles, eccrine, apocrine, and sebaceous glands. The axillary area represents a distinct ecological niche characterized by warm, moist, and nutritionally rich conditions. Salts, proteins, squalene, sterols, sterol esters, wax esters, a wide range of lipids and fatty acids are secreted, thus sustaining one of the highest densities of microorganisms on the body surface.

The human cutaneous microbiome plays an important role in the generation of bodily malodour, particularly in the underarm (axilla) region. A primary causal molecule of axillary malodour has been identified as the thioalcohol 3-methyl-3-sulfanylhexan-1-ol (3M3SH). 3M3SH is derived from biotransformation of an odourless dipeptide precursor (Cys-Gly-3M3SH) by certain bacterial species found in the axilla.

Gram-positive anaerobic cocci (GPAC) such as Anaerococcus octavius and Peptoniphilus spp. are a significant component of the axillary microbiome and show a strong association at the taxon level with underarm malodour intensity. Studies at the species level show that Staphylococcus hominis is positively associated with underarm malodour, whereas S. epidermidis and Cutibacterium acnes are anti-correlated.

S. epidermidis colonizes predominantly the axillae, head, and nares. Studies have shown that metabolic products of S. epidermidis, including organic acids such as lactic acid, improve skin moisture retention, maintain a low acidic condition on the skin surface, and improve rough skin texture. S. epidermidis is also considered to supplement the barrier function of the epidermis and inhibit the colonization of skin pathogens such as S. aureus through factors such as nutrient competition, production of antimicrobial peptides (AMPs), immunomodulatory properties (inhibition of inflammatory cytokine production) and enhanced expression of tight junction proteins.

C. acnes is another major skin commensal that prevents colonization and invasion of pathogens, via the hydrolysis of triglycerides in sebum and release of fatty acids that are antimicrobial and contribute to an acidic pH of the skin surface.

The main intervention strategy used for the treatment of armpit malodours is the topical application of deodorants and antiperspirants.

Although there is no doubt that treatments such as deodorants and antiperspirants are effective, they may also deplete or eliminate beneficial commensal microbial strains such as S. epidermidis and C. acnes. As noted above, these microbial strains are considered to play many important beneficial roles relating to skin health, skin appearance and skin function.

The present invention addresses this problem.

An objective of the present invention is to provide skin care benefits by microbial reprofiling of skin.

### SUMMARY OF THE INVENTION

The invention provides a cosmetic method for providing skin care benefits by microbial reprofiling of skin; the method comprising the steps of: (i) treating the skin by topical application of an antimicrobial metal salt, followed by (ii) washing the treated skin and treating the washed skin by topical application of a skin prebiotic active.

The invention also provides the cosmetic use of a skin prebiotic active, in combination with an antimicrobial metal salt, for providing skin care benefits by microbial reprofiling of skin.

### DETAILED DESCRIPTION OF THE INVENTION

In the context of this invention “skin” is understood as the layers which comprise it, from the uppermost layer or stratum corneum to the lowermost layer or hypodermis, both inclusive. These layers are composed of different types of cells such as keratinocytes, fibroblasts, melanocytes, mastocytes, neurons and/or adipocytes, among others.

As used herein, “skin care” means regulating and/or improving cosmetic qualities of the skin. These qualities are subject to regulation and/or improvement both in healthy subjects as well as those which present diseases or disorders of the skin (such as psoriasis, lichen planus, folliculitis, or atopic dermatitis).

Examples of skin care benefits in the context of this invention include reducing and/or preventing skin malodour; providing a smoother, more even texture; improving the elasticity or resiliency of the skin; improving the firmness of the skin; reducing the oily, shiny, and/or dull appearance of skin; improving the hydration status or moisturization of the skin, improving the appearance of fine lines and/or wrinkles; improving skin exfoliation or desquamation; plumping the skin; improving skin barrier properties; improving skin tone; reducing the appearance of redness or skin blotches and improving the brightness, radiancy, or translucency of skin.

A preferred skin care benefit in the context of this invention is reducing and/or preventing skin malodour, especially axillary malodour.

As used herein, the term “microbial reprofiling of skin” denotes selectively supporting the growth, metabolism and/or colonization of desirable microbial strains in the human cutaneous microbiome (such as S. epidermidis and C. acnes) in relation to other less desirable microbial strains in the human cutaneous microbiome (such as S. hominis, S. haemolyticus and S. lugdunensis). Collectively, these microbial strains naturally compete for local resources and attachment to epithelial sites. Rather than generally reducing microbial load and diversity, the compositions and methods of the invention render an ecological change that favours the growth, metabolism and/or colonization of the desirable over the less desirable microbial strains.

### Antimicrobial Metal Salt

Suitable antimicrobial metal salts for use in the invention act to decrease the density of the skin bacterial community.

Examples of antimicrobial metal salts for use in the invention include aluminium chlorohydrate (ACH), activated aluminium chlorohydrate (AACH), aluminium sesquichlorohydrate (ASCH), activated aluminium sesquichlorohydrate (AASCH), aluminium bromohydrate, aluminium chloride, aluminium sulfate, potassium aluminium sulfate, sodium aluminium chlorohydroxy lactate, aluminium zirconium chlorohydrate, zinc gluconate, zinc glycerinate, zinc acetate, zinc sulfate, zinc oxide, zinc citrate, zinc chloride, zinc pyrrolidone carboxylate, zinc lactate and zinc phenol sulfonate.

Mixtures of any of the above-described materials may also be used.

Preferred examples of antimicrobial metal salts for use in the invention are selected from aluminium chlorohydrate (ACH), activated aluminium chlorohydrate (AACH), aluminium sesquichlorohydrate (ASCH), activated aluminium sesquichlorohydrate (AASCH) and mixtures thereof.

### Skin Prebiotic Active

The term “skin prebiotic active” in the context of this invention denotes an ingredient that is metabolized by members of the skin microbiome and promotes microbial reprofiling of skin (as defined above).

Examples of skin prebiotic actives suitable for use in the invention are those which can selectively support the growth, metabolism and/or colonization of desirable microbial strains in the human cutaneous microbiome selected from S. epidermidis and/or C. acnes strains; whilst inhibiting, or not promoting, the growth, metabolism and/or colonization of less desirable microbial strains in the human cutaneous microbiome selected from S. hominis, S. haemolyticus and/or S. lugdunensis strains.

Such materials include pimelic acid, pimelic anhydride, sucrose, lactose, ribose, maltose, mannose, saccharide isomerate and glycerol.

Mixtures of any of the above-described materials may also be used.

Preferred examples of skin prebiotic actives for use in the invention are selected from pimelic acid, pimelic anhydride and mixtures thereof. Pimelic acid is a straight-chained, 7-carbon saturated a, w-dicarboxylic acid (IUPAC name heptanedioic acid).

In a preferred method according to this invention, step (i) is carried out on a first day of treatment, and step (ii) is carried out the following day and repeated daily for at least two further consecutive days thereafter (such as from two to ten days thereafter).

Suitably, a two-part cosmetic system for use in the method of this invention has a first composition comprising the antimicrobial metal salt in association with a second composition comprising the skin prebiotic active.

A first composition for use in the invention will generally contain from about 0.5% to about 30%, preferably from about 5 to 25% of antimicrobial metal salt (by weight based on the total weight of the composition). A preferred first composition for use in the invention will generally contain from about 0.5% to about 30%, preferably from about 5 to 25% (by weight based on the total weight of the composition) of antimicrobial metal salt selected from aluminium chlorohydrate (ACH), activated aluminium chlorohydrate (AACH), aluminium sesquichlorohydrate (ASCH), activated aluminium sesquichlorohydrate (AASCH) and mixtures thereof.

A second composition for use in the invention will generally contain from about 0.1% to about 15%, preferably from about 1 to 10% of skin prebiotic active (by weight based on the total weight of the composition). A preferred second composition for use in the invention will generally contain from about 0.1% to about 15%, preferably from about 1 to 10% of (by weight based on the total weight of the composition) of skin prebiotic active selected from pimelic acid, pimelic anhydride and mixtures thereof.

Compositions for use in the invention will generally include a cosmetically acceptable vehicle. The term “cosmetically acceptable” means that the vehicle is suitable for topical application to the skin, has good aesthetic properties, is compatible with the antimicrobial metal salt or skin prebiotic active respectively, and will not cause any safety or toxicity concerns. The cosmetically acceptable vehicles of the first and second compositions respectively may be the same or different.

Suitable cosmetically acceptable vehicles in the context of this invention may comprise an aqueous phase, an oil phase, an alcohol, a silicone phase, or a mixture thereof, and may be in the form of an emulsion. Emulsions can have a range of consistencies including thin lotions (which may also be suitable for spray or aerosol delivery), creamy lotions, light creams, and heavy creams.

Exemplary emulsions include water-in-oil emulsions, oil-in-water emulsions, silicone-in-water emulsions, water-in-silicone emulsions, polyol-in-silicone emulsions, silicone-in-polyol emulsions, polyol-in-oil emulsions, oil-in-polyol emulsions, wax-in-water emulsions, and water-oil-water triple emulsions. Preferred emulsions include oil-in-water emulsions and water-in-oil emulsions.

Compositions in the form of an emulsion, and suitable for use in the invention, typically have an oil phase containing at one or more cosmetically acceptable fatty materials which may be liquid or solid at room temperature (25° C.).

Suitable cosmetically acceptable fatty materials include naturally derived oils (such as sunflower oil, borage oil, soybean oil, castor oil, olive oil and almond oil); esters of monoalcohols or of polyols with monocarboxylic or polycarboxylic acids, at least one of the alcohols and/or acids comprising at least one hydrocarbon-based chain containing at least 6 carbon atoms (such as octyl palmitate, isopropyl myristate, isopropyl palmitate, isopropyl isostearate, hexyl laurate, isohexyl laurate, isohexyl palmitate, decyl oleate, isodecyl oleate, hexadecyl stearate, decyl stearate, dihexyldecyl adipate, lauryl lactate, myristyl lactate, cetyl lactate, oleyl stearate, oleyl oleate, oleyl myristate, lauryl acetate, cetyl propionate, isononyl isononanoate, propylene glycol dicaprate, diisopropyl adipate, dibutyl adipate, and oleyl adipate); ethers (such as dicapryl ether); fatty alcohols (such as cetyl alcohol, stearyl alcohol and behenyl alcohol); isoparaffins (such as isooctane, isododecane and isohexadecane); silicone oils (such as dimethicones, cyclic silicones, and polysiloxanes); and hydrocarbon oils (such as mineral oil, petrolatum and polyisobutene); fatty acids containing from 8 to 30 carbon atoms, (such as stearic acid, lauric acid, palmitic acid and oleic acid); vegetable fats (such as cocoa butter, coconut oil, palm oil and shea butter); petroleum-based, natural and synthetic waxes (such as lanolin wax, beeswax, carnauba wax, candelilla wax, paraffin wax, lignite wax, microcrystalline waxes, ceresin, ozokerite, and polyethylene waxes); hydrogenated oils which are solid at 25° C. (such as hydrogenated castor oil, hydrogenated jojoba oil, hydrogenated palm oil, hydrogenated tallow and hydrogenated coconut oil); and fatty esters that are solid at 25° C. (such as C₂₀-40 alkyl stearate).

Compositions in the form of an emulsion, and suitable for use in the invention, typically have an aqueous phase, which may also include one or more organic liquids that are miscible with water at room temperature (25° C.). Exemplary water-miscible organic liquids include monohydric and polyhydric alcohols and derivatives thereof such as C₂-C₆ alkanols (such as ethanol and isopropanol); C₂-C₁₀ glycols and polyols (such as glycerol, propylene glycol, butylene glycol, pentylene glycol, hexylene glycol, caprylyl glycol, dipropylene glycol, and diethylene glycol); C₃-C₁₆ glycol ethers (such as mono-, di-, or tripropylene glycol (C₁-C₄)alkyl ethers and mono-, di-, or triethylene glycol (C₁-C₄)alkyl ethers) and polyethylene glycol having 2 to 12 oxyethylene units.

Compositions in the form of an emulsion, and suitable for use in the invention, generally include surface active ingredients, such as emulsifiers and solubilizers, to enable two or more immiscible components to be combined homogeneously and to help stabilize the composition. Emulsifiers that may be used to form O/W or W/O emulsions include sorbitan oleate, sorbitan sesquioleate, sorbitan isostearate, sorbitan trioleate, PEG-20 sorbitan isostearate, polyglyceryl-3-diisostearate, polyglycerol esters of oleic/isostearic acid, polyglyceryl-6 hexaricinolate, polyglyceryl-4-oleate, polyglyceryl-4 oleate/PEG-8 propylene glycol cocoate, polyglyceryl-2 dipolyhydroxystearate, PEG-30 dipolyhydroxystearate, oleamide DEA, TEA myristate, TEA stearate, magnesium stearate, sodium stearate, potassium laurate, potassium ricinoleate, sodium cocoate, sodium tallowate, potassium castorate, sodium oleate, cetyl phosphate, diethanolamine cetyl phosphate, potassium cetyl phosphate, sodium glyceryl oleate phosphate, dimethicone copolyol, cetyl dimethicone copolyol, octyldimethicone ethoxyglucoside copolyol, dimethicone copolyol crosspolymer and laurylmethicone copolyol.

Compositions for use in the invention may also be formulated in a single-phase carrier such as a hydrophobic or hydrophilic liquid. Suitable hydrophobic liquid carriers include liquid polyorganosiloxanes, mineral oils, hydrogenated polyisobutene, polydecene, paraffins and isoparaffins of at least 10 carbon atoms, aliphatic or aromatic ester oils (such as isopropyl myristate, lauryl myristate, isopropyl palmitate, diisopropyl sebacate, diisopropyl adipate and C₁₂ to C₁₅ alkyl benzoates), polyglycol ethers (such as polyglycol butanol ethers) and mixtures thereof. Suitable hydrophilic liquid carriers include water, monohydric or polyhydric aliphatic alcohols having 2 to 8, preferably 2 or 3 carbon atoms (such as ethanol and isopropanol, oligoglycol ethers having 2 to 5 repeat units (such as dipropylene glycol) and mixtures thereof.

Liquid form compositions for use in the invention may be thickened, for example using one or more water soluble or colloidally water-soluble polymeric thickening agents. Suitable water soluble or colloidally water-soluble polymeric thickening agents include hydroxyethyl cellulose, methyl cellulose, hydroxypropyl methyl cellulose, polyquaternium-10, carrageenan, guar gum, hydroxypropyl guar gum, xanthan gum, polyvinylalcohol, acrylic acid/ethyl acrylate copolymers, carboxyvinyl polymers, cross-linked polyacrylate polymers and polyacrylamide polymers.

Solid form compositions for use in the invention, such as gels and sticks, generally include one or more structurants, in an amount ranging from about 1% to about 35% (by weight based on the total weight of the composition), depending upon the desired product consistency. Examples of structurants include fatty acid gelling agents selected from saturated fatty acids or hydroxy acids containing from about 8 to 30 carbon atoms and their salts, esters or amides (such as sodium and potassium stearate, 12-hydroxystearic acid, esters of 12-hydroxystearic acid and amides of 12-hydroxystearic acid); saturated, unsubstituted monohydric fatty alcohols containing from about 8 to 30 carbon atoms (such as cetyl alcohol, myristyl alcohol and stearyl alcohol); acyl amino acid derivatives such as N-lauroyl-L-glutamic acid di-n-butylamide; amide derivatives of di or tribasic carboxylic acids such as alkyl N,N′ dialkylsuccinimide; inorganic particulate thickening agents selected from siliceous and alumino-siliceous materials (such as fumed silicas, bentonites, hectorites, colloidal magnesium aluminium silicates and organoclays such as stearalkonium bentonite, disteardimonium hectorite, quaternium 90-bentonite), quaternium-18 bentonite and quaternium-18 hectorite); and petroleum-based, natural and synthetic waxes (such as lanolin wax, beeswax, carnauba wax, candelilla wax, castor wax, montan wax, paraffin wax, lignite wax, microcrystalline waxes, ceresin, ozokerite, polymethylene and polyethylene waxes).

Combinations of any of the above-described materials or product forms may also be used.

Compositions for use in the invention (as described above) may include additional skin care actives for improving the physical and/or aesthetic characteristics of the skin. Examples include vitamins, minerals and/or antioxidants, emollients, humectants, skin lightening agents, sunscreens, anti-irritants, exfoliating agents, herbal extracts (such as pomegranate, white birch, green tea, chamomile, and licorice extracts) and mixtures thereof.

Compositions for use in the invention (as described above) may include additional functional ingredients for improving the physical and/or aesthetic characteristics of the composition per se. Examples include inorganic pigments (such as titanium oxide, zirconium oxide, cerium oxide, zinc oxide, iron oxide, chromium oxide, manganese violet, ultramarine blue, chromium hydrate and ferric blue); organic pigments (such as carbon black and the organic lakes of barium, strontium, calcium or aluminium); pearlescent agents (such as mica coated with titanium oxide and/or iron oxide); dyes, preservatives (such as disodium EDTA, benzyl alcohol, methylparaben, phenoxyethanol, propylparaben, ethylparaben, butylparaben and isobutylparaben); pH adjusters and fragrances (such as essential oils, flower oils, natural extracts from resins, gums, balsams, beans, mosses and other plants, as well as synthetic aromatic materials).

Mixtures of any of the above-described materials may also be used.

### Packaging

Compositions for use in the invention (as described above) may be packaged in suitable containers to suit viscosity and intended use by the consumer. For example, a liquid composition can be packaged in a bottle or a roll-ball applicator, or in a container fitted with a pump suitable for finger operation, or in a propellant-driven aerosol device. Gel or cream compositions can be packaged in a non-deformable bottle or squeeze container, such as a tube or a lidded jar, or in an applicator having a dispensing head provided with at least one aperture through which the composition can be extruded under mild pressure. Solid compositions may be shaped into a stick form which may be elevated out of a dispensing package and directly applied to the desired area of skin.

The invention will be further illustrated by the following, non-limiting Example.

### Example

A two-part cosmetic system according to the invention was formulated with a first composition and a second composition. The first and second compositions are roll-on emulsions having ingredients as shown in Table 1a and Table 1b, respectively.

TABLE 1a

| Ingredient | wt. % |
|---|---|
| ACH | 12 |
| Steareth-2 | 2.6 |
| Glycerin | 4 |
| Steareth-20 | 0.6 |
| PPG-15 Stearyl ether | 4 |
| Water, antioxidant, chelating agent | to 100 |

TABLE 1b

| Ingredient | wt. % |
|---|---|
| Pimelic acid | 5 |
| Helianthus annuus seed oil | 4 |
| Steareth-2 | 3 |
| Steareth-20 | 0.8 |
| Sodium hydroxide | 1 |
| Water, antioxidant, preservative | to 100 |

An in vivo human study was conducted to evaluate the performance of the cosmetic system on axillary malodour and the axillary microbiome.

The first composition (Table 1a) was applied after washing to both underarms of healthy test subjects on day 1 of the study. The second composition (Table 1b) was applied after washing to one underarm of the test subject in 3 consecutive daily doses (on days 2, 3 and 4 of the study, respectively) with the other underarm serving as a control (washed but no second composition applied).

Mean malodour score (MMS) of the control and treated underarms was assessed on a daily basis by a team of expert odour assessors, with results shown in Table 2.

TABLE 2

| MMS | Treated | Control | Difference | LSD | p value |
|---|---|---|---|---|---|
| day 2 | 2.411 | 2.353 | −0.058 | 0.213 | 0.5799 |
| day 3 | 2.287 | 2.41 | 0.123 | 0.25 | 0.329 |
| day 4 | 2.344 | 2.548 | 0.204 | 0.202 | 0.048 |

The results demonstrate that the treatment according to the invention leads to a progressive and significant reduction in mean malodour score compared to the control.

Microbiological sampling of the control and treated underarms (via swabs) was also carried out on a daily basis. The results demonstrated that the treatment according to the invention leads to a reprofiling of the axillary microbiome with a selective reduction in malodour associated species compared to the control.

1. A cosmetic method for providing skin care benefits by microbial reprofiling of skin; the method comprising the steps of: (i) treating the skin by topical application of an antimicrobial metal salt, followed by (ii) washing the treated skin and treating the washed skin by topical application of a skin prebiotic active, in which the antimicrobial metal salt is selected from aluminium chlorohydrate (ACH), activated aluminium chlorohydrate (AACH), aluminium sesquichlorohydrate (ASCH), activated aluminium sesquichlorohydrate (AASCH) and mixtures thereof, and in which the skin prebiotic active is selected from pimelic acid, pimelic anhydride and mixtures thereof, and in which the skin care benefit is reducing and/or preventing axillary skin malodour.

2. A method according to claim 1, in which step (i) is carried out on a first day of treatment, and step (ii) is carried out the following day and repeated daily for at least two further consecutive days thereafter.

3. A two-part cosmetic system for use in a method according to claim 1, the system having a first composition containing from 5 to 25% of an antimicrobial metal salt by weight based on the total weight of the first composition in association with a second composition containing from 1 to 10% of a skin prebiotic active by weight based on the total weight of the second composition in which the first composition contains from 5 to 25% by weight based on the total weight of the first composition of antimicrobial metal salt selected from aluminium chlorohydrate (ACH), activated aluminium chlorohydrate (AACH), aluminium sesquichlorohydrate (ASCH), activated aluminium sesquichlorohydrate (AASCH) and mixtures thereof; and the second composition contains from 1 to 10% by weight based on the total weight of the second composition of skin prebiotic active selected from pimelic acid, pimelic anhydride and mixtures thereof.

4. (canceled)
